(19) United States
(12) Patent Application Publication
(10) Pub. No.: US 2015/0065919 A1
(43) Pub. Date: Mar. 5, 2015

(54) POSTURE TRAINING DEVICE

(71) Applicants: Jose Antonio Cuevas, San Juan, PR (US); Eduardo A Carbia, San Juan, PR (US)

(72) Inventors: Jose Antonio Cuevas, San Juan, PR (US); Eduardo A Carbia, San Juan, PR (US)

(21) Appl. No.: 14/011,053

(22) Filed: Aug. 27, 2013

(51) Int. Cl. A61B 5/11; A61B 5/00

(52) U.S. Cl. CPC A61B 5/1116; A61B 5/1121; A61B 5/1126; A61B 5/6823; A61B 5/746
USPC 600/587

(57) ABSTRACT

A posture training device for monitoring posture is disclosed. The posture training device comprises at least a first measuring circuit unit attached to a first position on a user's body, and a second measuring circuit unit attached to a second position on the user's body. Each measuring circuit unit comprises an accelerometer, gyroscope, and magnetometers. Further the accelerometers, gyroscopes, and magnetometers generate a signal received by a computing platform unit. Any improper posture is indicated to the user by an alarm signal generated by the computing platform unit.

### RELATED APPLICATIONS

This application is related to provisional U.S. Patent Application No. 61/693,415 filed on Aug. 27, 2013.

### STATEMENT REGARDING FEDERALLY SPONSORED RESEARCH AND DEVELOPMENT

N/A

### BACKGROUND OF THE DISCLOSURE

1. Field of the Disclosure

The present disclosure relates generally to a non-invasive posture training device that continually monitors a user's spine, more particularly, a posture training device that measures the back's position and trains an individual to maintain movement patterns to ensure a particular posture.

2. Discussion of the Background

Some well-known devices include devices capable of obtaining data which basically comprises means for receiving signals from sensors, means to store data related to sensors' signals, and means to communicate one device with other external devices.

The use of already existing devices for obtaining data in order to control posture is influenced by various factors. The first factor is the use of only accelerometers for calculating the angles relative to the spinal column or backbone by ignoring the drift in the orientation over time, and thus progressively producing measurement errors from any tilting, bending, and straightness. Another factor is the use of analogical sensors which results in mayor distortions in the captured signals.

Further, another factor is the size and energy consumption of a device, thus limiting working hours, increasing the device's surface, and hindering its ergonomics. Also another factor is the connection to other equipment through wired serial protocols with wires instead of wireless, thus allowing the user to move freely without being attached to anything. Similarly another factor is the lack of enough memory for data storage that allows subsequent analysis and an efficient use of the device for much more time. Furthermore another factor is the number of sections of the back to be analyzed for a correct study of all the possible postures by using 3 points to measure.

Yet another factor is the stimulus response that is done with strict parameters configured, without integrating any artificial intelligence system to recognize new correct or incorrect positions.

Therefore, there is a need to provide an apparatus and method for back posture training that overcomes the disadvantages and shortcomings of the prior art.

### SUMMARY

In general, the present disclosure overcomes the disadvantages and shortcomings of prior art by disclosing a non-invasive device and a method for obtaining related data with one of more parameters that are detected through inertia measurement units, such as orientation sensors. Accordingly, it is an object of the present disclosure to provide a safe to use circuit with sensors for capturing the parameters related to the spine position, and other anatomical parts of the body wherein said sensors are capable of sending digital signals to a computing platform device. The computing platform device receives and uses a sequence of instructions to generate and store the data related to the spine position. Further, if needed, the data can be provided to the users at a different point from where the data is captured or measured for additional analysis.

The exemplary embodiment in accordance with the principles of the present disclosure comprises several low energy consume modules for data acquisition working in real time finding a more precise orientation in regard to the other tools for posture control. The present modules and structure conceives greater precision in taking measurements and to real time capturing and analysis of tilting, bending, and straightness; twist thus providing more independence and interdependence in the data transfer of each segment to be analyzed, and improving its versatility in choosing a particular function.

It is another object of the present disclosure to provide means to store data obtained by the inertia measurement units. In accordance with the principles of the present disclosure inertia measurement units such as accelerometers, gyroscopes, and magnetometers is incorporated to the sensor's circuit.

Still another object of the present disclosure is to provide the integration of a tri-axial accelerometer, a tri-axial gyroscope, and a tri-axial magnetometer per each segment to be analyzed. In accordance with the principles of the present invention the non-invasive posture training device will monitor and control the relation between the values of the three zones. Only multiple, interrelated sensors can monitor and control lateral deviations and a twisted posture.

Another object of this disclosure is to provide means to control the communication with the sensors and external devices. The inertia measurement units are capable of receiving data are digital, thus avoiding any type of analogical/digital conversion.

Another object of this disclosure is to provide means to control posture correction for aesthetics and work related purposes.

Still another object of the present disclosure is to provide posture correction monitoring device for clinical purposes; back pain, spine abnormality, rehab, prescribed treatment, etc. Another object of this disclosure is to provide a monitoring and recording device for pre and post-surgery posture correction and measurement with quantitative results.

Another object of this disclosure is to provide means to control posture by recording in various daily activities for diagnosis of ailments.

Another object of this disclosure is to provide means to control Monitoring, recording and corrective device for compliance with physician's instructions.

Still another object of the present disclosure is to monitoring and posture correction device for sports and performance enhancement.

It is another object of this disclosure to provide means for biofeedback. The current disclosure comprises a process that enables an individual to learn how to change physiological activity for the purposes of improving health and performance.

Accordingly, it is an object of the present disclosure to provide Preventive back pain instrument. The current disclosure provide means to store the information received by the computation platform device which is used to measures three axes, in the three movable sectors of the spine, creating a virtual axis and triangulations. As each sensor becomes the vertex of a triangle, it will also monitor the related anatomical structure of the pelvic and scapular waist.

In accordance with the principles of the present disclosure comprises a computing platform device comprising a microprocessor, wherein said microprocessor comprises a sequence of instructions to process in real time the data received from the inertia measurement units. Therefore, it is another object of this disclosure to provide means to control, analyze and generate alerts for incorrect positions. In addition the non-invasive posture training device can provide a true 3D dynamic assessment and monitoring of the spine.

Further in accordance with the principles of the present disclosure the exemplary embodiment comprises a sensor holder, wherein said sensor holder fixes at least three different inertia measurement units to a particular back zone. The sensor holder is configured to consider the ergonomics and form of the spine.

The disclosure itself, both as to its configuration and its mode of operation will be best understood, and additional objects and advantages thereof will become apparent, by the following detailed description of a preferred embodiment taken in conjunction with the accompanying drawings.

The Applicant hereby asserts, that the disclosure of the present application may include more than one invention, and, in the event that there is more than one invention, that these inventions may be patentable and non-obvious one with respect to the other.

Further, the purpose of the accompanying abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the disclosure of the application, which is measured by the claims, nor is it intended to be limiting as to the scope of the disclosure in any way.

### BRIEF DESCRIPTION OF THE DRAWINGS

The accompanying drawings, which are incorporated herein, constitute part of the specification and illustrate the preferred embodiment of the disclosure.

FIG. 1 shows a general structure connection for the measuring system in accordance with the principles of the present disclosure.

FIG. 2 shows a general structure connection for the computing platform device in accordance with the principles of the present disclosure.

FIG. 3 shows a general structure of the sensor holder first exemplary embodiment connected wireless external device in accordance with the principles of the present disclosure.

FIG. 4 shows a flowchart exemplary embodiment of the back position training in accordance with the principles of the present disclosure.

FIGS. 5A-5B shows a first exemplary embodiment of the Graphical unit interface in accordance with the principles of the present disclosure.

FIG. 6 shows a first exemplary embodiment of the data stored in accordance with the principles of the present disclosure.

FIG. 7 shows a first exemplary more detailed circuit for the measuring units in accordance with the principles of the present disclosure.

FIG. 8 shows a first exemplary embodiment of the computing platform device in accordance with the principles of the present disclosure.

FIG. 9 shows an exemplary embodiment of the series of circuits for the measuring units connected to the computing platform device in accordance with the principles of the present disclosure.

FIG. 10 shows a first exemplary embodiment of the non-invasive sensor holder in accordance with the principles of the present disclosure.

FIG. 11A-11D. show side and top views of the alert activation process for the first exemplary embodiment in accordance with the principles of the present disclosure.

FIG. 12A-12B show back views of the alert activation process for the first exemplary embodiment in accordance with the principles of the present disclosure.

FIG. 13A-13B. shows more detailed views of the sensor holder for the first exemplary embodiment in accordance with the principles of the present disclosure.

FIG. 14 shows a detailed view of the second exemplary embodiment of the non-invasive sensor holder in accordance with the principles of the present disclosure.

FIG. 15 shows an exemplary embodiment of the belt in accordance with the principles of the present disclosure.

FIG. 16 shows an exemplary embodiment of the top circuit sensor housing in accordance with the principles of the present disclosure.

FIG. 17 shows a second exemplary embodiment of the middle circuit sensor housing in accordance with the principles of the present disclosure.

FIG. 18 shows a second exemplary embodiment of the of the computing platform device in accordance with the principles of the present disclosure.

FIG. 19 shows a second exemplary embodiment of the non-invasive sensor holder attached to an user in accordance with the principles of the present disclosure.

FIG. 20 shows a third exemplary embodiment of the non-invasive sensor holder in accordance with the principles of the present disclosure.

FIG. 21A-21B. show side views of the alert activation process for the third exemplary embodiment in accordance with the principles of the present disclosure.

FIG. 22A-22B show back views of the alert activation process for the third exemplary embodiment in accordance with the principles of the present disclosure.

FIG. 23A-23B show more detailed views of the sensor holder for the third exemplary embodiment in accordance with the principles of the present disclosure.

### DESCRIPTION OF THE PREFERRED EMBODIMENT

The system determines angles in real time in regard to each of the three (3) segments of the backbone in the X, Y, and Z components in order to calculate the tilting of each axis. However, an approximation for calculating the tilting of each axis can be performed by directly integrating some accelerometers, but with the passing of cycles the orientation will drift and result in an orientation error and thus its correct calculation. In order to avoid this situation and obtain a precise orientation over time, it is necessary to integrate through an algorithm various inertia measurements units, such as accelerometers, gyroscopes, and magnetometers (9 degrees of freedom). This way one can efficiently avoid the possible drifts that happen over time, thus generating a very stable system. The system will then require several sensors for each region of the back to be analyzed.

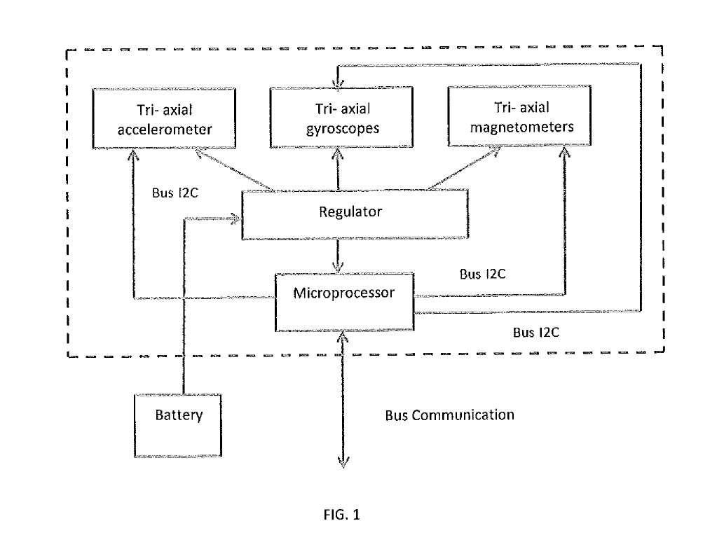

Referring to FIG. 1, the general structure connection for the measuring system in accordance with the principles of the present disclosure comprises measuring units, wherein said measuring units comprises accelerometers, gyroscopes, and magnetometers. The measuring units are in communication with the microprocessor through a switching unit or regulator. Further an energy source is coupled to the regulator for powering the measuring system. A more detailed structure is explained below.

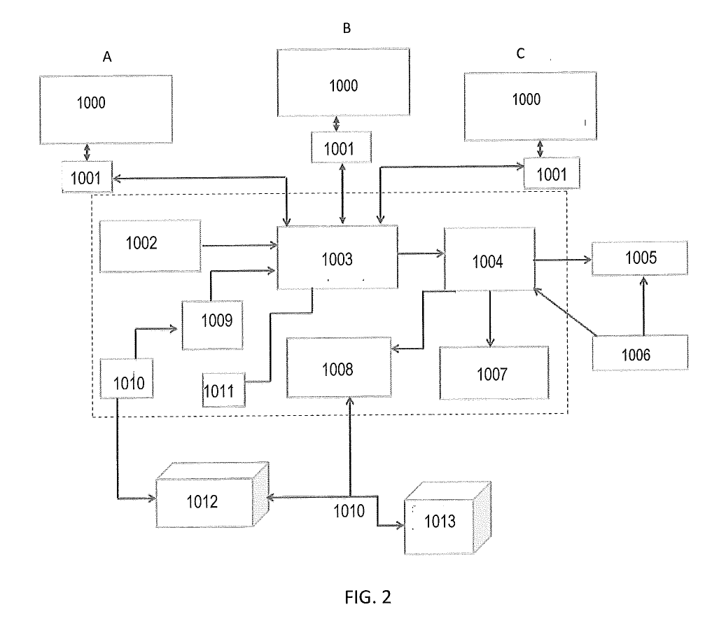

FIG. 2 shows a general structure connection for the computing platform device in accordance with the principles of the present disclosure. Elements of FIG. 2 are defined as follow:
A Cervical Zone
B Thoracic Zone
C Lumbar Zone
1000 9DOF Circuit
1001 MP
1002 Database (Micro SD)
1003 Micro-controller
1004 Expert System
1005 Display
1006 Parameters Controller
1007 Vibration Motor
1008 Bluetooth
1009 Battery
1010 USB
1011 Clock
1012 PC
1013 Smartphone Tablet

The principal characteristics of the computing platform device are: the capacity to acquire effectively different types of digital signals, for example angles in X, Y, Z (tri-axial) for the accelerometer, gyroscope, and magnetometer) by integrating them in an algorithm in order to obtain the precise orientation of a rigid body; and the capacity to analyze the correct postures from the incorrect ones by generating an alert in the form of a vibrating or audible stimulus, by integrating an expert system capable of analyzing the new guidelines and generating an ideal response.

Further the computing device can be connected to a computer or other device (Smartphones, Tablets, etc. . . . ) through a USB port or even Bluetooth in order to share stored data. In addition to its adjusted intake in performance, that allows the design and creation of a system for the determination of complex data with a simple system of energy input.

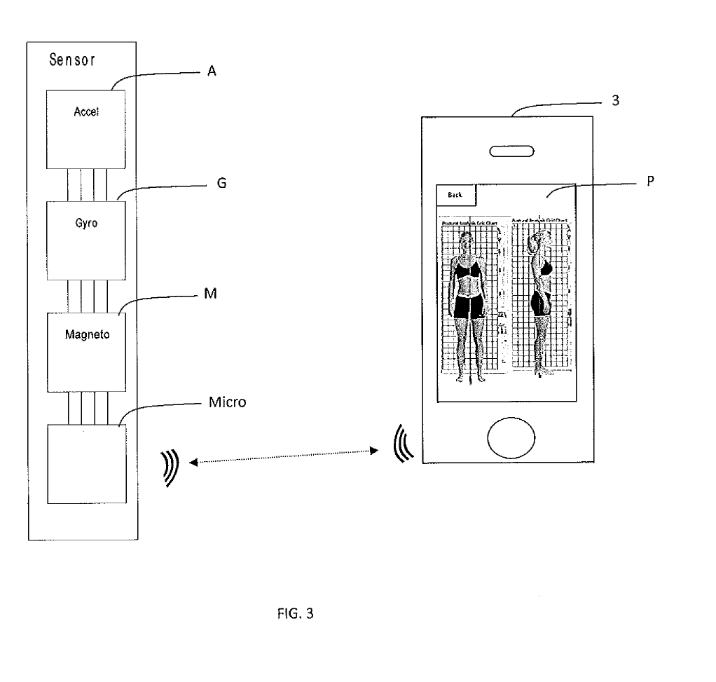

The computing platform device system has a microcontroller, for example an AVR architecture microcontroller as nucleus. The microcontroller may comprise an internal flash memory and EEPROM memory, internal as well, for data storage. This microcontroller and the developed program that guides the system's operation allows sharing store information through direct communication with a computer through the USB port as well as wireless communication through Bluetooth as shown in FIG. 3. This communication uses a piece of hardware to translate data between parallel and serial form, for example a UART (Universal Asynchronous Receptor-Transmitter) standard. The control of the feeding is done by a circuit in commutation that allows a 3.3 Vdc highly stable tension to exist out of a battery.

The communication among the posture training device PTD components is configured to be digital, thus reducing the interferences and the need for using an analogical-digital converter. In addition, the data from each group of sensors is obtained independently through a processor per group. The processor can follow the I2C protocol if desired.

Brief Description of the Steps to Follow with the Method of Using the Current Disclosure:

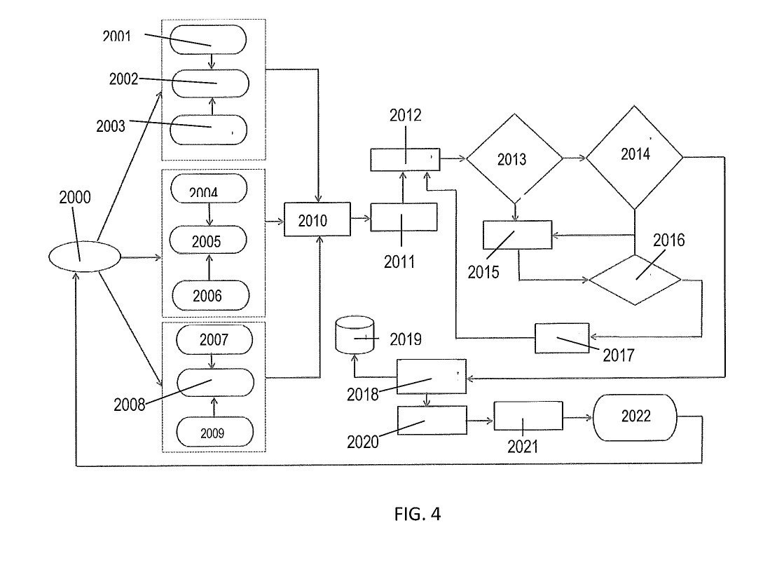

Elements of FIG. 4 are defined as follow:
2000 Start
2001 Cervical Accelerometer
2002 Cervical Gyroscope
2003 Cervical Magnetometer
2004 Thoracic Accelerometer
2005 Thoracic Gyroscope
2006 Thoracic Magnetometer
2007 Lumbar Accelerometer
2008 Lumbar Gyroscope
2009 Lumbar Magnetometer
2010 Integration of measurements
2011 Estimate Angels/deviations
2012 Compare Segments
2013 Is the deviation per segment between the tolerance ranges?
2014 Is the deviation per segment among
2015 Generate Alert
2016 Corrected back position?
2017 Rules System
2018 Upload data of measurements into the device in real time and
2019 DB
2020 Transmit data
2021 Other devices data reception
2022 Display and analysis of the acquired data

As shown in FIG. 4, each group comprises at least an accelerometer, a gyroscope and a magnetometer. Each group of sensors is provided with a sequence of instructions. For example a sequence of instructions based on the direction cosine matrix (DCM) that continuously uses a 3×3 rotation matrix, defining the roll (r), the pitch (p), and the yaw (y), which are the angles with respect to a fixed axis in reference to the Earth's directions.

DCM is a product matrix of other three individuals describing the rotation over axis “X”, axis “Y,” and axis “Z.” Gyroscopes measure the rotation over these axes in the backbone's framework. Accelerometers measure the axes' acceleration over the plane, and the magnetometers measure the magnetization with respect to such axes.

The information obtained from the sensors will define 3 vectors, one for each sensor, being able to organize it in a matrix of sensors. The DCM algorithm will consist of four stages:
a. Matrix Update:

Means a multiplication matrix of DCM with the integration matrix of gyres.
b. Normalization.
c. Correction of deviations.
d. Euler angles.

This way one will determine the Euler angles in each axis within the backbone's framework.

Process of the Performance Example:

The system will individually analyze the different regions of the backbone or part of the body to be analyzed and the relations among them as a global system. In the instance case the back is selected as the body part to be analyzed. In the selected example, the system is divided into 3 specific zones (Lumbar Zone, Thoracic Zone, and Cervical Zone), although able to add more to the system without modifying the hardware, only the number of sensor groups into which one would like to divide the backbone is available. The placement of the sensors for this example has as an objective the analysis of the principal mobile curvatures of the back, thus placing each group of sensors in the following way: Cervical C1-T1; Thoracic T1-L1; Lumbar L1-L5.

Each group of sensors will be placed in a mobile curvature of the backbone, in order to obtain information in real time and transmit it to the corresponding microprocessor, which will perform a first filtering and organization of the obtained data, thus making it available to be sent to a main microprocessor. This main microprocessor has been programmed to integrate the obtained data through the sensors in the previously described algorithm and to be able to obtain the Euler angles of each zone of the backbone to be analyzed, being able to correctly identify the group of sensors.

The set of rules described in the main microprocessor for the analysis of the obtained data will allow the user to adjust the parameters as necessary for each concrete situation, being able to vary the correct or incorrect deviation intervals, the time necessary for producing a stimulus, or even the stimulus time. In addition, the integration of an expert system will allow automatic variations in those rules, thus achieving more efficiency in the system.

If any of the curvatures is great or flat, then the backbone is showing deviations, making necessary their individual analysis or the analysis of existing interdependence among them. By controlling the 3 curvatures of the backbone, one can efficiently avoid bad postures and abnormalities such as lumbar lordosis and kyphosis. In the same way, muscular tension and pinching produced by an incorrect curvature of these 3 movable sections of the back that guide the correct behavior for the control of posture.

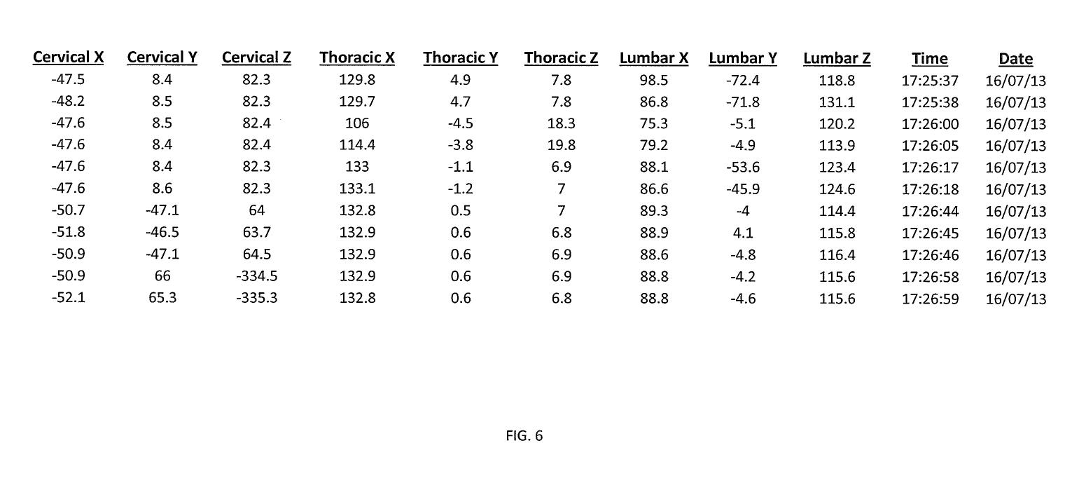

The posture control device aims to maintain the natural curve of the backbone in order to prevent muscular or biomechanical problems that cause pain or any other related pathology. The computing platform device for the control of posture helps to facilitate therapeutic study and analysis, monitoring in real time the posture of the back and its 3 curvatures independently or relative to each other, thus producing a vibrating or audible stimulus when bad posture is detected in the patient. For example see FIG. 6. Consequently, the device users will be able to learn good posture habits that will benefit them for the rest of their lifetime.

The control of vibrating stimuli will be guided by some rules described by specialists in the field of area of the part to be analyzed such as a specialist in the field of back pain diagnoses, along with artificial intelligence systems, thus allowing improvements in the control of stimuli and a more specific and individual monitoring for each user and pathology. The rules are converted into codes representing pre-determined values to be recognized by the posture training device, more particularly the computing platform device. The pre-determined values are further compare with a current back position during the back posture analysis.

The non-invasive posture training device includes a clock in order to control data storage in an organized way and to analyze in a more coherent way the obtained results; since the computing platform device is wireless and will be able to be used during daily tasks, a micro SD memory has been included, thus allowing a greater quantity of data to be determined during a long period of time. This is why the device does not only work as a learning system, but it also works as a method for studying the adopted postures by following the parameters required by the specialist.

The parameters of the device will be able to be controlled through the main computing platform device by observing the options to select in the display and thus configuring the different parameters as desired, for example pre-determined values. For example a graphical unit interface (GUI) G1, G2 can be provided for this purpose.

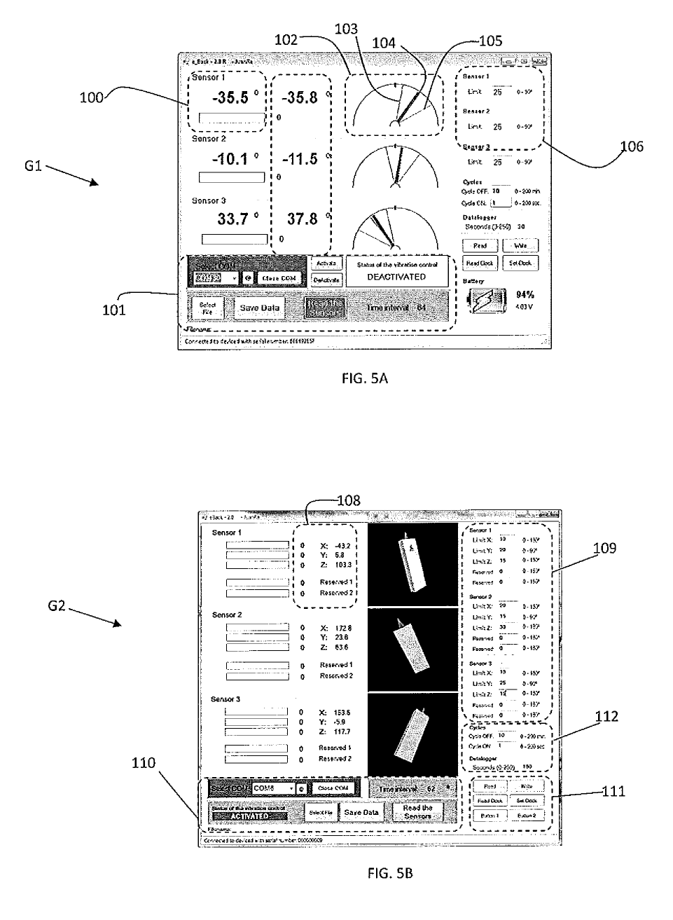

In accordance with the principle of the present disclosure the main computing platform device obtains data in real time, for each of the 3 sensors and the 3 position angles at the part of the body, in the instance case the back. As show in FIG. 5A through 5B, with these measures, based on the reference of the desired posture, the non-invasive posture training device verifies that these are within the valid set ranges. The positions are then measured in angles for each of the three axes (X, Y and Z). The permissible range is also set in angles. To configure these settings and the angles measured in real time by the device, a set of instruction is used. For example, a single axis GUI G1 can be provided in order to study or present a particular deviation. In order to set up a range a range input 106 is provided by the GUI G1. The current measure is presented digitally 100 and as a graphic 102 for visual purposes. The pre-determinated limited range 103 is also included as part of the graphic in such way that the GUI user can identify the current posture 104, more particularly the high limit range 103 and lower limit range 105. Several options are provided as part of the single axis GUI G1 in order to activated or control the data acquisition, manipulation of information and activation of the alarm device. Further the same information is used for a more complex purpose. Each measure axis of each sensor is combined to provide current readings 108 with a 3D graphic as part of a multiple axes GUI G2. Further the GUI G2 can include a column 109 for the configuration of parameters of the non-invasive posture training device PTD. On one side are the ranges for each sensor that are allowed before activating the incorrect posture warning and vibration. On one section of the GUI G2 are a time input 112 for setting up the number of seconds allowed to be outside the range of the correct position before activating the warning and the vibration. Every time the system activates, it records all the related information such as time, position, degrees of deviation, etc. To change the values, the user will type the new ranges. If done correctly, a message will appear on the bottom. It is important to understand that the set of instructions at least analyzes three different variables (X,Y,Z) but more variables may be introduced for posture analyzing purposes. For example temperature or other variable which result in including at least a temperature sensor to each circuit 1a.

The most important characteristics of the circuits are the following:
Group of sensors:
   Digital communication following I2C protocol.
   Microcontroller incorporation.
   Low-consumption required feeding.
   Design minimization.
   Tri-axial accelerometer, tri-axial gyroscope, and tri-axial magnetometer integration.
   Integrated circuits for each tri-axial accelerometer, tri-axial gyroscope, and tri-axial magnetometer.
programmed main microcontroller:
   Internal memory storing the sequence of instructions.
   Capability to store the device configurations.
   Fast transmission of data.

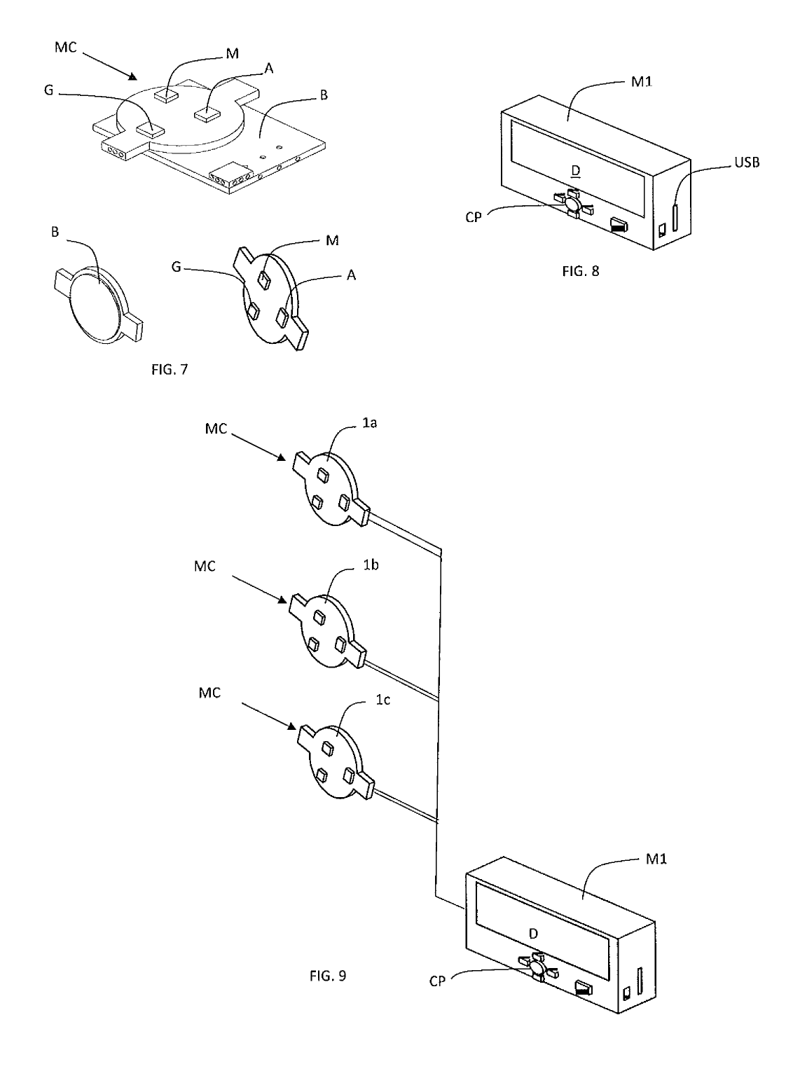

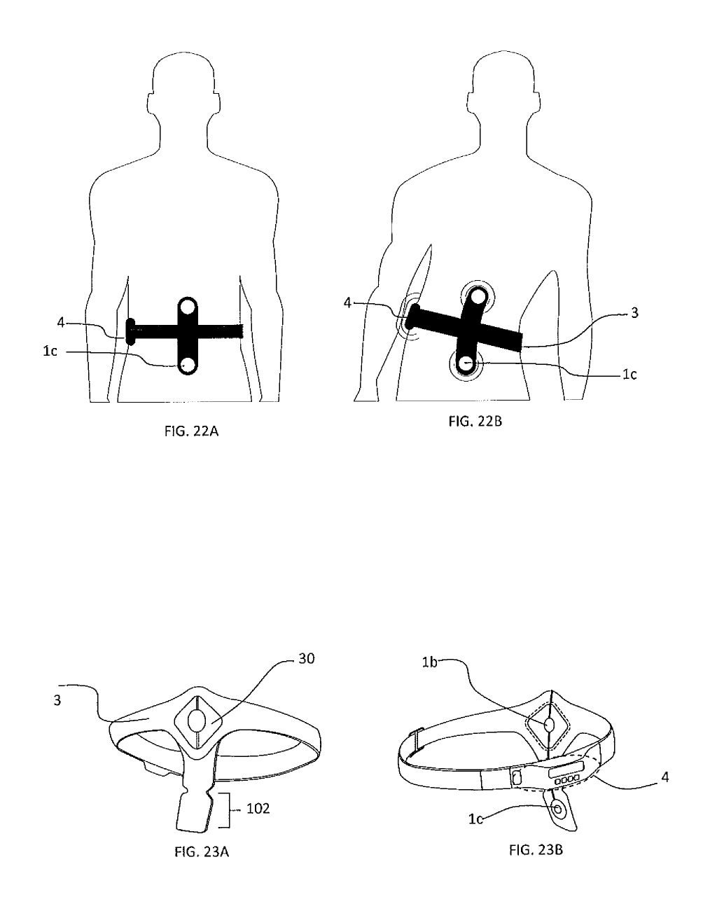

The integration of the sensors to the users can be made in various ways, such as by sticking them to the indicated zone with adhesive pads and soft cables, by integrating them to adjustable harnesses according to the anthropology of the individual, or by integrating them into t-shirts. Below is the discussion of a preferred embodiment. The computing platform device is desired to be integrated into a protective case, such as a box, for the protection of its components, and in reference to the global system, it may be adhered in various ways; whether by integrating in the harness or by pasting onto a belt. An exemplary embodiment in accordance with the principle of the current invention is provided from FIG. 7 through FIG. 22.

The sequence of instructions that is used for the configuration of the computing platform device for the control of posture is the posture training system comprises different configuration options in order to work with different types of future versions, thus becoming a clear and simple interface for easy interpretation and handling by the specialists and the users.

The principal configuration options are the following:
Port speed: the speed of the communications port is selected.
Value-indicating window: the acquired value of the selected zone is shown.
System configuration: comprises the selection of the desired parameters for the analysis, as time intervals for the stimulus or difference in degrees, individual selection of the segment or of the global system.
Software control panels: where one can initiate the communication, stop it, store the data obtained by the system, Bluetooth connection, and exit.
Real-time visualization window of the backbone's behavior.
Diagnostics window.
Indicating window for the incorrect postures adopted during the analysis.

The non-invasive posture training device PTD structure, as show in FIG. 7 through FIG. 23 for controlling of posture can be presented in two formats of reduced dimensions, one for measuring unit circuit sensors MC and the computing platform device M1, wherein said computing platform device M1 comprises the main microprocessor.

In FIG. 7 the circuit sensor MC comprises at least an accelerometer A, at least a gyroscope G and a least a magnetometer M mounted on a circuit board B. FIG. 8 is directed to the computing platform device M1. The computing platform device M1 comprises a Universal serial bus USB connection for sharing information and/or connecting to computers, as well as, for recharging the posture training device's batteries. In addition, computing platform device M1 comprises a display D that guides one in the selection of parameters that one requires and in the response to the generated stimuli. In reference to the collection of sensors, as shown in FIG. 9, each circuit sensor MC is coupled wired or wireless to the computing platform device M1.

Each circuit sensor MC and said computing platform device M1 are coupled to a posture training device PTD. The posture training device PTD, as shown in FIG. 9 is ergonomic, safe to use and adjustable for each user 2. The first exemplary embodiment for the posture training device PTD comprises a center bar 1 and a belt 3 with attachable and adjustable means, such as conventional buckle and a snap latch, as in U.S. Pat. No. 5,862,526 here included by reference. Further, said center bar extends from said belt 3.

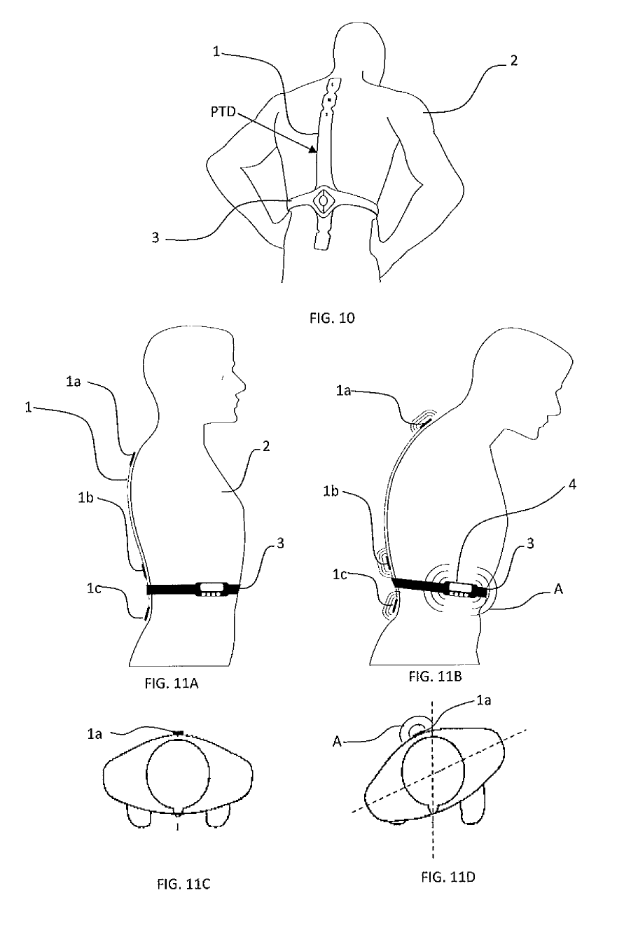

The center bar 1 comprises a top circuit sensor 1a configured to be located at the Cervical zone, a middle circuit sensor 1b configured to be located at the thoracic zone and a bottom circuit sensor 1c configured to be located at the lumbar zone. Wherein the posture training device PTD detect a defected posture or a posture away from the set up parameter an alert AL is generated by the computing platform device 4 as disclosed above. The defected motion is detected in at least three different dimensions per circuit sensor MC. For example a defected frontal posture, as shown in FIG. 11B, a twisted torso posture, as shown in FIG. 11D, and/or a side defected posture as shown in FIG. 12B.

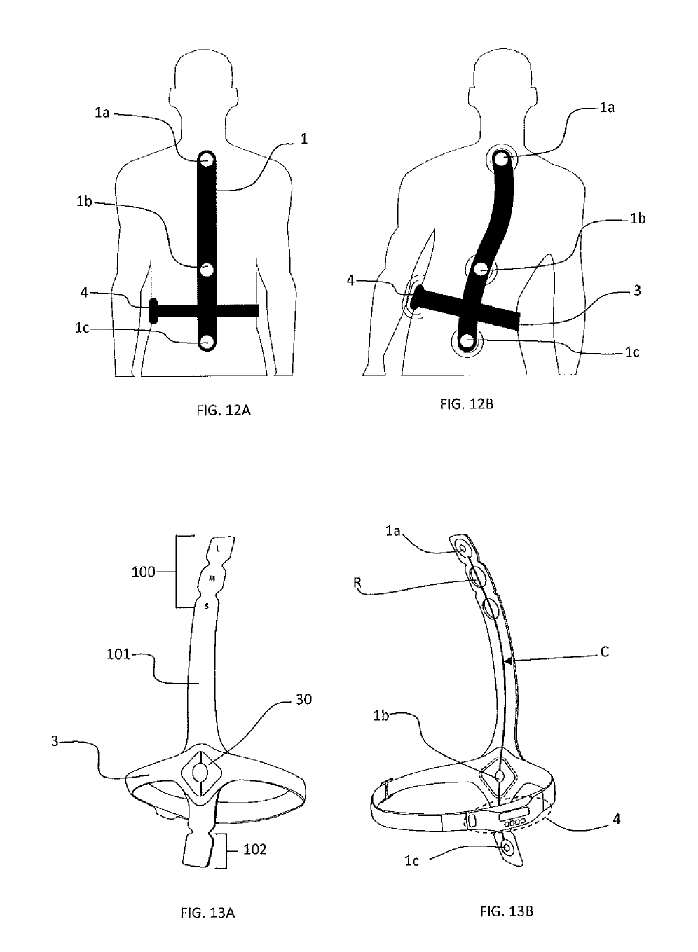

The center bar 1 comprises a top section 100, a middle section 101 and a bottom section 102, as shown in FIG. 13A through FIG. 13B. The inner part of the center bar 1 comprises recesses R in order to locate the circuit sensors 1a, 1b, 1c. The top section 100 comprises at least several recesses R partially close, to each other, as shown in FIG. 12B, providing different extensions of the top circuit 1a from the belt 3 depending on the selected recess R.

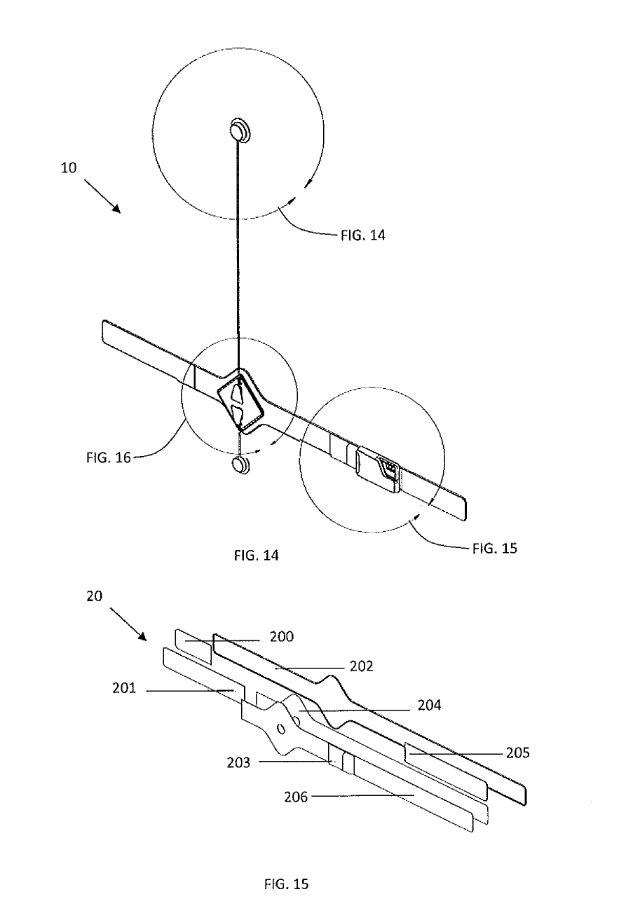

A second embodiment 10 of the posture training device is shown from FIG. 14 through FIG. 18. The second embodiment is quite similar from the first embodiment but instead of the center bar 1 comprises extendable cable 304 which extends from a belt 20. The belt 20, as shown in FIG. 15, comprises an attaching mean 200, a receiving mean 205, an elastic member 201, a battery pocket 203, a mesh fabric 204 and a synthetic rubber 206. The attaching mean 200 and receiving mean 205 are used to attach the belt 20 to the user. The attaching mean 200 comprises a velcro-hook while the receiving mean 205 comprises a Velcro loop.

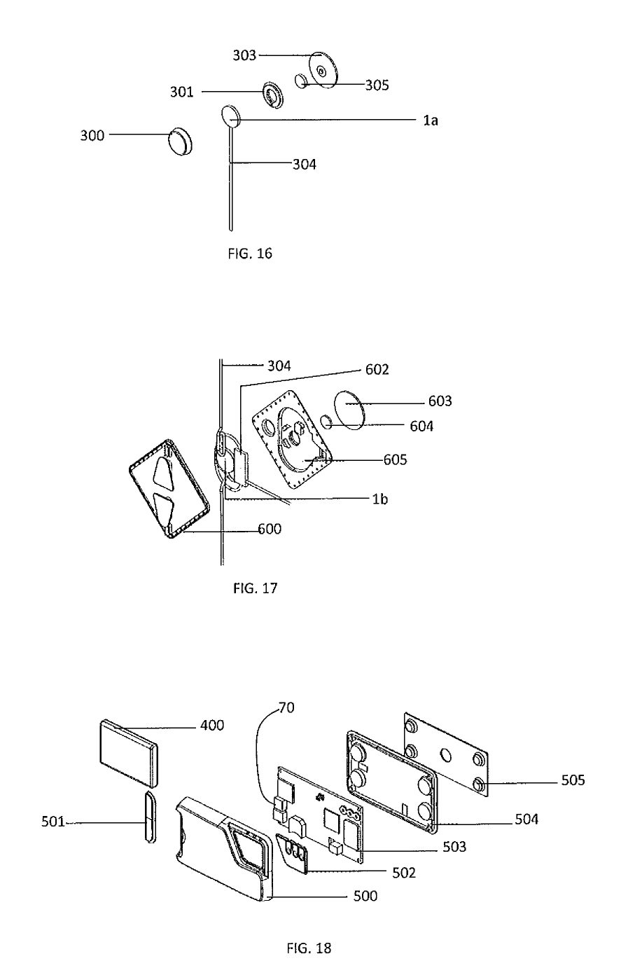

FIG. 16 is directed to the top circuit sensor and bottom circuit holder housing 30. The circuit sensor holder housing 30 comprises a top cap 300, an extendable cable 304 electrically coupled to circuit sensor 1a, a bottom cap 301, a patch connector 305 and an adhesive skin patch 303. The extendable cable 304 connects the circuit sensor 1a to the computing platform device 50.

FIG. 17 is directed to the circuit sensor holder housing 60 for the middle circuit 1b. The middle circuit sensor holder housing 60 comprises a top cap 600, a co-processor 602 electrically coupled to circuit sensor 1b, a bottom cap 605, a patch connector 604 and an adhesive skin patch 603. The circuit sensor 1b to the computing platform device 50 by means of the co-processor 602 is electrically coupled to the computing platform device 50.

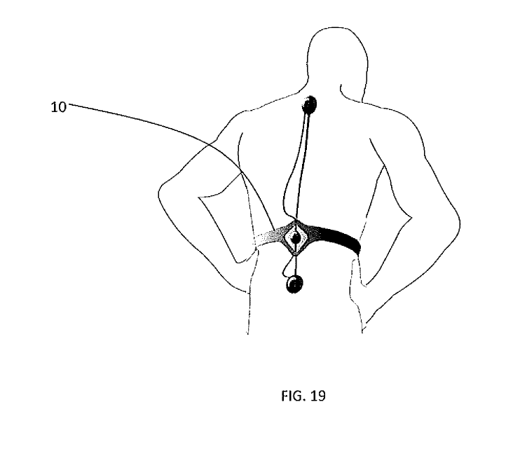

FIG. 18 is directed to the computing platform device 50 comprising a PC board 503 and bottom cap 505, a top cap 500, a plastic buttons 502, and a ports cap 501. The plastic buttons are provided for contacting purposes in order to manually control the computing platform device through the device inputs or control panel. The board 503, as mentioned before comprises a microprocessor and features to store data. Further the board 503 comprises several ports 70. All the circuit sensors are electrically connected to the computing platform device 50 in order to receive sensors signals. A battery 400 serves as a power supply, however other alternatives, such as solar power supply, can be used as long it provides enough energy to energize the computing platform device 50. FIG. 19 is directed to a user wearing the second embodiment 10.

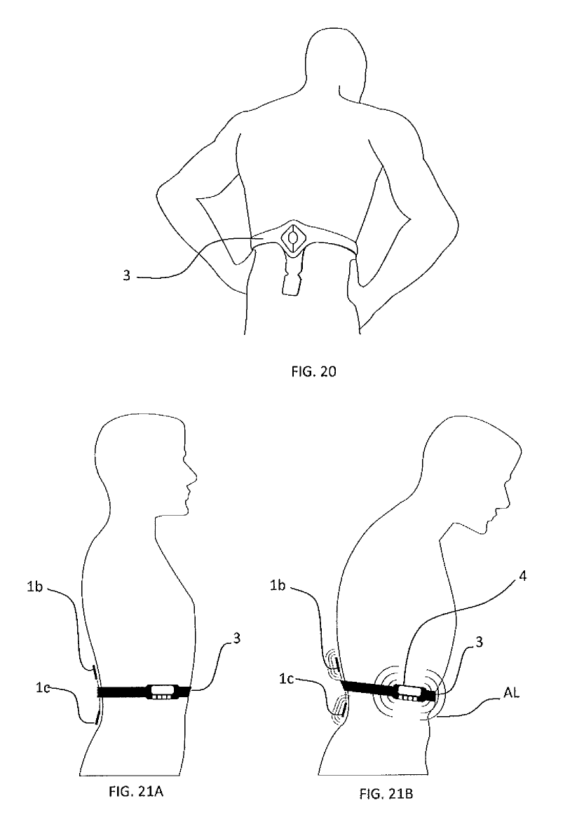

A third embodiment of the posture training device is shown from FIG. 20 through FIG. 23B. The third embodiment similar from the first comprises the same elements but instead of three circuit sensors MC just use two.

With the control panel CP, one can select using a input signal device, such as a keyboard, touch screen or any other means that translate information into a code accepted by the computing platform device 4 the desired parameters or pre-determined value, such as the specific control of a group of referenced sensors to a concrete segment of the backbone or the selection of a determined interval, or the specific time for any parameter to be analyzed.

The Led will appear turned on when the device is recharging its batteries, and as soon as it is turned off the batteries will be completely charged.

Good posture will be indicated as a correct position of the segments and the body in one moment and as a determined activity.

The disclosure is not limited to the precise configuration described above. While the disclosure has been described as having a preferred design, it is understood that many changes, modifications, variations and other uses and applications of the subject disclosure will, however, become apparent to those skilled in the art without materially departing from the novel teachings and advantages of this disclosure after considering this specification together with the accompanying drawings. Accordingly, all such changes, modifications, variations and other uses and applications which do not depart from the spirit and scope of the disclosure are deemed to be covered by this disclosure as defined in the following claims and their legal equivalents. In the claims, means-plus-function clauses, if any, are intended to cover the structures described herein as performing the recited function and not only structural equivalents but also equivalent structures.

All of the patents, patent applications, and publications recited herein, and in the Declaration attached hereto, if any, are hereby incorporated by reference as if set forth in their entirety herein. All, or substantially all, the components disclosed in such patents may be used in the embodiments of the present disclosure, as well as equivalents thereof. The details in the patents, patent applications, and publications incorporated by reference herein may be considered to be incorporable at applicant's option, into the claims during prosecution as further limitations in the claims to patently distinguish any amended claims from any applied prior art.

1. An apparatus, comprising:
a plurality of measuring circuit units comprising at least a first measuring circuit unit and a second measuring circuit unit;
a computing platform;
an adjustable supporting device;
wherein each measuring circuit unit comprises a circuit board with multiple variable measurement sensors; and
wherein said supporting device comprises at least a body part attaching structure and extendable structure, wherein said extendable structure is configured to be positioned on a user's body and receives at least said first measuring circuit.

2. The apparatus of claim 1, wherein each measuring circuit unit comprises a accelerometer generating a first signal, gyroscope generating a second signal, and magnetometer generating a third signal.

3. The apparatus of claim 2, wherein each measuring circuit unit comprises a first microprocessor receiving the first signal, second signal, and third signal.

4. The apparatus of claim 3, wherein said computing platform comprises a second microprocessor receiving a first microprocessor signal.

5. The apparatus of claim 3, wherein said computing platform comprises;
a data base to store at least the first signal, second signal, and third signal;
a clock to control the information stored in the database;
output for sharing information with external device;
a set of instructions for comparing a first microprocessor's signal with a pre-determined value;
and an stimulus generating element to generate a stimulus.

6. The apparatus of claim 5, wherein said stimulus is selected from a group of audible stimulus.

7. The apparatus of claim 5, wherein said stimulus is selected from a group of vibration stimulus.

8. The apparatus of claim 5, wherein said means for comparing the first microprocessor's signal comprises a second microprocessor.

9. The apparatus of claim 5, wherein said output for sharing information with external device comprises means for direct communication.

10. The apparatus of claim 5, wherein said output for sharing information with external device comprises means for wireless communication.

11. The apparatus of claim 5, wherein said output for sharing information with external device comprises means for wireless communication a data storage device.

12. Learning device for the control of the posture comprising:
multiple inertia measurement units for determining angles and orientations;
means for storing data from the inertia measurement units;
at least one integrated circuit;
and a support structure for receiving the inertia measurement units, wherein said posture device structure comprises attaching structure for holding the inertia measurement units at particular body parts;
and wherein each inertia measurement unit comprises at least two different sensors use for motion determination.

13. The learning device of claim 12, wherein each inertia measuring unit comprises a first microprocessor receiving a first signal from an accelerometer sensor and a second signal generated from the gyroscope sensor.

14. The learning device of claim 13, wherein said first microprocessor receives a third signal generated from the magnetometer sensor.

15. The learning device of claim 12, wherein said microcontroller comprises a second microprocessor receiving a first microprocessor's signal.

16. The learning device of claim 15, wherein said microcontroller comprises;
a data base to store at least the first signal and second signal;
a clock to control the information stored in the database;
means for sharing information with an external device;
means for comparing a first microprocessor's signal with a pre-determined value;
and means to generate a stimulus.

17. The learning device of claim 16, wherein said stimulus is selected from a group of audible stimulus or vibration.

18. The learning device of claim 16, wherein said means for comparing the first microprocessor's signal comprises a second microprocessor.
